(19) United States
(12) Patent Application Publication
(10) Pub. No.: US 2003/0162227 A1
(43) Pub. Date: Aug. 28, 2003

(54) Soluble, freely diffusible Sonic hedgehog protein

(76) Inventors: David J. Robbins, Cincinnati, OH (US); John A. Goetz, Cincinnati, OH (US); Xin Zeng, Chestnut Hill, MA (US)

Correspondence Address: DINSMORE & SHOHL, LLP, 1900 CHEMED CENTER, 255 EAST FIFTH STREET, CINCINNATI, OH, 45202, US

(21) Appl. No.: 10/377,361

(22) Filed: Feb. 28, 2003

Related U.S. Application Data
(60) Provisional application No. 60/360,322, filed on Feb. 28, 2002.

(51) Int. Cl. G01N 33/53; C07K 14/47

(52) U.S. Cl. 435/7.1; 530/350

(57) ABSTRACT

Isolated native forms of soluble, freely diffusible Sonic hedgehog protein and recombinant forms of soluble, freely diffusible oligomeric Sonic hedgehog protein are produced. Methods of assaying an oligomeric Sonic hedgehog protein derived from a tissue and/or cell comprise measuring a conditioned media to detect the production of the soluble, freely diffusible Sonic hedgehog protein.

### GOVERNMENT INTERESTS

This invention was made, at least in part, with funds from the Federal Government, awarded through grant number R01CA82628-01. The U.S. Government therefore has certain acknowledged rights to the invention.

### FIELD OF THE INVENTION

The present invention is directed towards isolated native forms of soluble, freely diffusible Sonic hedgehog protein and to recombinant forms of soluble, freely diffusible oligomeric Sonic hedgehog protein. The present invention is also directed towards methods of assaying a Sonic hedgehog protein derived from a tissue and/or cell. The methods comprise measuring a conditioned media to detect the production of the soluble, freely diffusible Sonic hedgehog protein.

### BACKGROUND OF THE INVENTION

The hedgehog family (Hh) of secreted proteins play an important role in the patterning of a diverse array of animals. Hh was first identified as a Drosophila Melanogaster segment polarity gene required for embryonic patterning. Hh also plays an essential role in the development of various Drosophila adult organs, regulating the patterning of the various imaginal discs that eventually form adult structures. Hh family members have also been implicated in the patterning of numerous vertebrate structures, an observation underscored by the dramatic phenotypes of animals mutant for the various Hh family members. Hedgehog is expressed in specific regions of the animal termed organizing centers, which influence patterning of surrounding tissues through both short- and long-range signaling activity.

Hedgehog has three homologues in humans; Desert, Indian and Sonic hedgehog (Shh). It has been proposed that Hh family members can act as morphogens, inducing discrete cell fates in a concentration-dependent manner. Previous research has indicated that Hh proteins can induce various concentration-dependent cell fates in vitro; this role however has been controversial in vivo. This controversy arose because these early studies used a form of Shh not found in native tissues, which lacked the lipid modifications found on wild-type Shh. The lipid modifications on wild-type Shh facilitate its association with the plasma membrane, raising the question of how Shh is capable of moving far away from its site of synthesis to act as a morphogen. Additionally, previous research has used carboxy-cleaved Sonic hedgehog to support the simple diffusion model of long-range Sonic hedgehog signaling.

Accordingly, there is a substantial need to develop novel improved forms of Sonic Hedgehog protein, and particularly forms which are soluble and freely diffusible, and preferably biologically active.

### SUMMARY OF THE INVENTION

Accordingly, it is an object of the invention to provide novel forms of the Sonic hedgehog protein. Another object of the invention is to provide novel Sonic hedgehog proteins which are freely diffusible and biologically potent.

In accordance with one embodiment, the invention is directed to an isolated native form of soluble, freely diffusible Sonic hedgehog protein (s-shhNp).

In accordance with another embodiment, the invention is directed to a recombinant form of soluble, freely diffusible Sonic hedgehog protein. This form of the protein is oligomeric.

In accordance with yet another embodiment, the invention is directed towards methods of assaying a Sonic hedgehog protein derived from a tissue and/or cell. The methods comprise measuring a conditioned media to detect the production of the soluble, freely diffusible Sonic hedgehog protein.

In accordance with yet another embodiment, the invention is directed towards methods for identifying compounds which increase or inhibit the production of soluble, freely diffusible Sonic hedgehog protein (Shh). The methods comprise expressing oligomeric Shh in the presence of a test compound and comparing an amount of oligomeric Shh expressed in the presence of the compound with an amount oligomeric Shh expressed in the absence of the compound.

Additional embodiments, objects and advantages of the invention will become more fully apparent in view of the following detailed description.

### BRIEF DESCRIPTION OF THE DRAWING

FIG. 1 illustrates the pathway for Sonic hedgehog protein to directly induce long-range biological effects.

### DETAILED DESCRIPTION

One of ordinary skill in the art will appreciate the various utilities derived from novel forms of the Sonic hedgehog protein. These utilities include, but are not limited to, embryonic patterning and regulating the patterning of adult structures, including, but not limited to, adult organs and vertebrate structures.

The secreted protein Sonic hedgehog (Shh) exerts many of its patterning effects through a combination of short and long range signaling. Three distinct mechanisms, which are not necessarily mutually exclusive, have been proposed to account for the long-range effects of Sonic hedgehog: simple diffusion of Sonic hedgehog; a relaying mechanism in which Sonic hedgehog activates the secondary signals; and direct delivery of Sonic hedgehog through cytoplasmic extensions, termed cytonemes. As used herein, the term “s-ShhNp” refers to oligomeric Sonic hedgehog protein, including recombinant/expressed and native forms.

Shh, like Hh, is produced as an approximate 45 kDa precursor protein that subsequently undergoes an intramolecular cleavage reaction to generate an amino-terminal polypeptide (ShhNp, p stands for processed) and a carboxyl terminal polypeptide (ShhC). During this cleavage cholesterol is covalently attached to the last amino acid of the amino-terminal polypeptide. This cleavage event occurs in an undefined intracellular compartment prior to the secretion of both ShhNp and ShhC. In developing the present invention, the inventors recognized that the initial investigations of the properties of these two cleavage products of Shh revealed that, while ShhNp was almost exclusively membrane associated, ShhC was freely diffusible. However, previous research has provided insignificant evidence for a native or expressed form of Shh that is freely diffusible and not membrane-associated.

The present inventors have expressed freely diffusible form of Shh (s-ShhNp) that is cholesterol modified, oligomeric and biologically potent. Further, the inventors have determined that the availability of s-ShhNp is regulated by two functional antagonists of the Shh pathway, Patched (Ptc) and Hedgehog-interacting protein (Hip). Finally, the inventors have determined a gradient of s-ShhNp across the anterior-posterior axis of the chick limb, demonstrating the physiological relevance of s-ShhNp.

Specifically, the inventors have expressed a soluble form of Sonic hedgehog (s-ShhNp) which demonstrates that Hh family members can exert many of their long-range effects directly. This form of Shh was extracted from the conditioned media of cells expressing full-length Shh. The conditioned media, when assayed in a cell-based C3H10T1/2 alkaline phosphatase induction assay, has a specific activity at least thirty times greater than that of conditioned media generated from Shh aminoexpressing cells. This freely diffusible form of Shh is cholesterol modified, as a radiolabeled form was extracted from cells metabolically labeled with [3H] cholesterol.

A native cholesterol-modified freely diffusible form of s-ShhNp was also isolated from a native source of Shh, the ZPA of chick limb duds. Additionally, an anterior-posterior gradient of s-ShhNp activity was detected across the limb bud. This gradient of activity extended beyond that detected with Shh antibodies, but correlated well with the domain of Shh-activated target genes. Thus, while not wishing to be bound by theory, the inventors believe that s-ShhNp may be responsible for the long-range signaling observed in the limb bud.

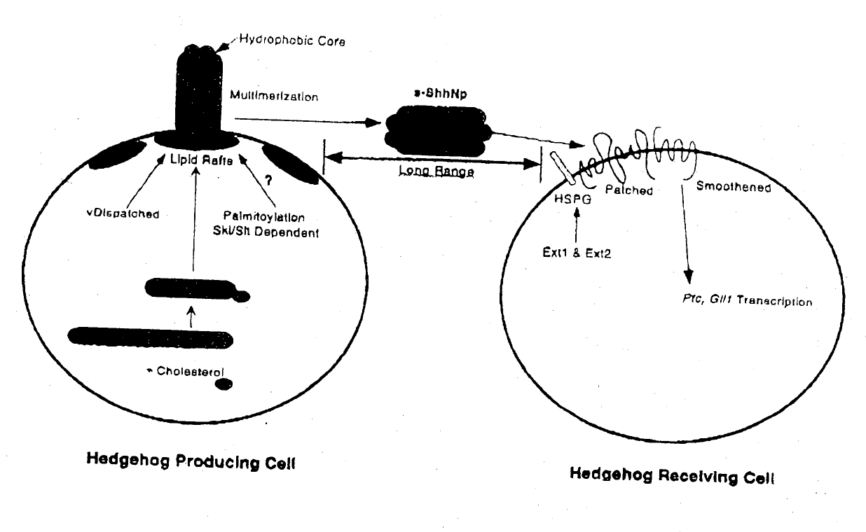

In isolating and expressing s-ShhNp, the inventors determined that in order for Hh proteins to exert their biological effects, they must first undergo multiple levels of post-translational processing. This processing includes an intramolecular cleavage that generates two discrete proteins, the addition of cholesterol and possibly palmitate to the amino terminal fragment, and the formation of the oligomeric, i.e., s-ShhNp. As depicted in FIG. 1, the inventors, while not wishing to be bound by theory, illustrate one possible way for Shh to directly induce long-range biological effects. Following Shh autocleavage, the amino-terminal fragment becomes cholesterol-modified and is targeted to lipid rafts. ShhNp becomes palmitoylated prior, or subsequent, to enrichment in lipid rafts. Presumably, this palmitoylation occurs in a manner dependent on mammalian Ski/Sit, and may help regulate s-ShhNp formation. The cholesterol-modified Shh, which has been enriched in lipid rafts, then forms mulitmers. Mulitmerization of ShhNp allows it to overcome its inherent hydrophobicity, by burying its hydrophobic regions into an adjacent ShhNp molecule. S-ShhNp diffuses away from the Shh-producing cells, at least in limb buds, to form a concentration gradient. The freely diffusible s-ShhNp then binds to Ptc to initiate Shh signaling; the strength of which would depend on the fractional occupancy of Ptc by Shh.

While not wishing to be bound by theory, the inventors further believe mammalian homologues of the Drosophila protein Dispatched (DISP) are involved in either the packaging of s-ShhNp or the targeting of ShhNp to lipid rafts, given that amorphic disp mutants are insensitive to HhNp. Additionally, the inventors recognize that Tout velu (Ttv), an enzyme involved in heparin sulphate biosynthesis, has been implicated as a necessary component in Hh-receiving cells. That is, Ttv mutations are insensitive to HhNp but responsive to HhN, suggesting that Hh's lipid modifications are necessary for Ttv to exert its normal biological effects. While not wishing to be bound by theory, the inventors believe that Ttv homologues may regulate the biosynthesis of a molecule that is necessary to recognize s-ShhNp but is not necessary for signaling by ShhN.

Given the proposed role of Ttv in Hh transport, while not wishing to be bound by theory, the inventors have determined that in vivo s-ShhNp may only be freely diffusible in a transient manner. Thus, a distinct subset of HSPGs, whose synthesis is regulated by the Ext family enzymes, may regulate the biosynthesis of a molecule that is necessary to recognize s-ShhNp.

In accordance with these determinations, the inventors have isolated a native form of soluble, freely diffusible Sonic hedgehog protein (s-ShhNp) and a recombinant form of soluble, freely diffusible oligomeric Sonic hedgehog protein. The isolated native and the oligomeric forms of the Sonic hedgehog are freely diffusible, lipid modified, and biologically active. The protein is biologically active in its ability to induce alkaline phosphatase. Furthermore, the s-ShhNp has a greater ability to induce alkaline phosphatase as compared with a nonoligomeric expressed Sonic hedgehog.

In addition, the inventors have developed methods of assaying an oligomeric Sonic hedgehog protein derived from a tissue and/or cell. The methods comprise measuring a conditioned media to detect the production of the soluble, freely diffusible Sonic hedgehog protein.

One skilled in the art will recognize the various properties that may be measured to determine if a conditioned media contains the soluble, freely diffusible Sonic Hedgehog protein. In one embodiment of the invention, the measurement comprises detecting the ability of the conditioned media to produce increased transcription of the genes for Patched, Glil or combinations thereof. Furthermore, one skilled in the art will recognize that various methods employed for determining the oligomeric state of the Sonic hedgehog protein may be used. In one embodiment of the invention, the oligomeric state of the Sonic hedgehog is determined by size-exclusion chromatography.

Furthermore, the inventors have developed methods for identifying compounds which increase or inhibit the production of oligomeric Sonic hedgehog protein (Shh). The methods comprise expressing oligomeric Shh in the presence of a test compound and comparing an amount of oligomeric Shh expressed in the presence of the compound with an amount oligomeric Shh expressed in the absence of the compound.

Specifically the methods provide therapeutic value as the methods can determine if drugs are able to increase or inhibit the production of the biologically active s-ShhNp. Cells, tissues samples, or animals treated with such drugs can be evaluated to determine the specific effects of the drug on the production of the freely diffusible s-ShhNp.

The foregoing description of the various embodiments of the invention has been presented for the purposes of illustration and description. It is not intended to be exhaustive or to limit the invention to the precise form disclosed. Many alternatives, modifications and variations will be apparent to those skilled in the art of the above teaching. Accordingly, this invention is intended to embrace all alternatives, modifications and variations that have been discussed herein, and others that fall within the spirit and broad scope of the claims.

### EXAMPLE

The following example demonstrates the properties of oligomeric Sonic hedgehog and the methods for assaying an oligomeric Sonic hedgehog derived from a tissue and/or cell.

Bosc 23 cells are transfected with constructs encoding full-length Sonic hedgehog protein and a control vector (ShhN). The conditioned media from the various transfected Bosc cells is added to C3H1OT1/2 cells to measure Sonic hedgehog biological activity. The conditioned media from Bosc cells transfected with full-length Shh differentiated the C3HIOT1/2 cells and has activity comparable to media from ShhN-transfected cells. The lipid-modified form of Shh (ShhNp) is cell-associated whereas s-ShhN, which is not lipid modified, was mostly soluble.

s-ShhNp is Lipid Modified

To determine the amount of s-ShhNp, immunoprecipitation is carried out, followed by immunoblotting with Shh antibodies. A small amount of soluble Shh from full-length Shh-conditioned media and a large amount of ShhN from ShhN-conditioned media is detected. S-ShhNp migrates at the same apparent molecular weight (on a SDS-polyacrylamide gel) as ShhNp from lysates of transfected Bosc cells and faster than ShhN, consistent with it being modified by cholesterol.

s-ShhNp is Soluble

The Shh-conditioned media is then subjected to ultracentrifugation, to rule out the possibility that s-ShhNp was only Shh-bound to membranous vesicles. The sShhNp is divided into two aliquots. One aliquot is set aside, while the other is centrifuged at 100,000 g for 120 min. Any resulting pellet is resuspended with an equal volume of media. There is no change in alkaline phosphatase (AP) activity between the centrifuged and non-centrifuged conditioned media, demonstrating that the Shh in the media is indeed soluble.

In an additional method, to address how s-ShhNp, a protein with two lipophilic modifications, is soluble in an aqueous environment conditioned media is isolated from cells transfected with full-length Shh or ShhN and subjected it to analysis by gel filtration chromatography. These conditioned media are isolated under serum-free conditions in which they are still biologically active to verify that sShhNp is not just binding to proteins present in serum. s-ShhNp migrated at about six times its native molecular weight. In contrast, ShhN migrated through this column close to its predicted molecular weight.

s-ShhNp is Oligomeric

To determine whether Shh could multimerize with itself to form the oligomeric Shh complex, s-ShhNp is immunoprecipitated from the conditioned media of cells co-transfected with Shh and a Flag-tagged Shh construct. Antibodies to the Flag epitope are able to coimmunoprecipitate untagged Shh, demonstrating that Shh multimerizes with itself.

s-ShhNp Biological Activity is Derived from Sonic Hedgehog protein

To determine if the biological activity in the Shh-conditioned media is due to Shh, three distinct assays of Shh activity are used: activation of AP in C3H1OT1/2 cells; activation of Ptc transcription in C3H1OT1/2 cells; and activation of a Gliluciferase reporter in Shh-Light2 cells”. These assays are performed in the presence or absence of two specific Shh inhibitors—the Shh neutralizing monoclonal antibody 5El and the teratogen cyclopamine. In the AP assay, aliquots of conditioned media are pre-incubated with the 5E1 monoclonal antibody or an isotype-matched control, then added to C3H1OT1/2 cells, followed by AP staining (FIG. 2a). The biological activity of the Shh-conditioned media is almost completely inhibited by the 5El monoclonal antibody, but unaffected by immunoglobulin-γ (gamma) (IgG). S-ShhNp is able to activate Ptc transcription in a manner comparable to ShhN and this activation is inhibited by cyclopamine, as is induction of AP. Furthermore, s-ShhNp activates Shh-Light2 cells approximately tenfold more than control conditioned media, and most of this activity is specifically inhibited by the 5El monoclonal antibody. Thus, the biological activity of the Shh-conditioned media is due to sShhNp, as two mechanistically distinct antagonists of the Shh pathway can inhibit the activity of the conditioned media in both direct and indirect assays of Shh activity.

To examine the effects of Shh receptor Ptc and Hip antagonists on the biological activity of s-ShhNp cells with Shh and Ptc, Hip or a vector control are cotransfected, and then the conditioned media is tested for biological activity. Conditioned media from Shh-transfected cells produced in the presence of Ptc or Hip has reduced biological activity compared with media from Shh co-transfected with a vector control. This decrease in biological activity correlates with decreased s-ShhNp in the conditioned media from Ptc- or Hip-expressing cells. Thus, Ptc and Hip, two physiologically relevant inhibitors of the Shh pathway, are able to reduce the activity of s-ShhNp in the conditioned media by reducing its availability.

Potency of s-ShhNp

To compare the potency of ShhN to s-ShhNp, Bosc cells are transfected with Shh, ShhN or a vector control, then the various conditioned media is collected. 50% dilution of the ShhN-conditioned media has comparable biological activity to full-strength s-ShhNp conditioned media. However, comparable amounts of s-ShhNp and ShhN immunoreactivity are only seen when ShhN media was diluted thirty times, indicating that s-ShhNp is greater than fifteen times more biologically potent than ShhN.

Isolation of s-ShhNp in Chick Limb-Bud System

To verify that s-ShhNp may be isolated from a physiologically relevant source of Shh the chick limb-bud system is investigated. Similar-sized fragments of posterior limb-bud tissue are dissected out and cultured in DMEM to allow s-ShhNp to diffuse out. Conditioned media from posterior regions of the limb bud strongly induced AP activity, and this biological activity is blocked by the 5El monoclonal antibody and cyclopamine. Conditioned media from posterior tissue is also able to activate Shh-Light2 cells 3-4 times over that of control media. Additionally, s-ShhNp can be detected in immunoprecipitates from posterior tissue conditioned media, but is absent from media conditioned in the absence of limb tissue. This endogenous form of soluble Shh migrates, on SDS-PAGE, in a manner consistent with it also being cholesterol modified. These results demonstrate that s-ShhNp can also be obtained from a physiologically relevant source of Shh, the posterior region of chick limb buds.

To investigate the presence of a soluble Shh gradient across the limb bud, similar-sized pieces of posterior, central or anterior tissue are incubated in tissue culture media to isolate s-ShhNp. Under conditions in which the AP assay is linear, strong Shh activity is obtained from posterior tissues, approximately 5-6 times less activity is obtained from central tissues, and even less Shh activity is obtained from anterior regions of the limb bud. Most of the s-ShhNp activity obtained from all regions of the limb bud is blocked by the Shh inhibitory monoclonal antibody. Polymerase chain reaction after reverse transcription of RNA (RT-PCR) demonstrates that Shh transcription is not upregulated in limb tissue after culture. Thus, s-ShhNp is produced from a physiologically relevant source of Shh, the limb bud, where it forms a gradient from posterior regions across the midline of the limb bud and even into more anterior regions of the limb bud. While not wishing to be bound by theory, although the data shows a gradient of s-ShhNp across the limb bud, additional factors inducing low levels of Shh expression in our reporter cell line may be implicated.

The specific embodiment and example set forth above is provided for illustrative purposes only and are not intended to limit the scope of the following claims. Additional embodiments of the invention and advantages provided thereby will be apparent to one of ordinary skill in the art and are within the scope of the claims.

What is claimed is:

1. An isolated native form of soluble, freely diffusible Sonic hedgehog protein.

2. A recombinant form of soluble, freely diffusible oligomeric Sonic hedgehog protein.

3. The protein according to claim 2, wherein the protein is operable to induce alkaline phosphatase.

4. The protein according to claim 3, wherein the protein has a greater ability to induce alkaline phosphatase as compared with a nonoligomeric Sonic hedgehog protein.

5. A method of assaying an oligomeric Sonic hedgehog protein derived from a tissue and/or cell comprising measuring a conditioned media to detect the production of the soluble, freely diffusible Sonic hedgehog protein.

6. A method according to claim 5, wherein the measuring step comprises detecting the ability of the conditioned media to increase transcription of genes for Patched, Glil, or combinations thereof.

7. A method according to claim 5, wherein an oligomeric state of the Sonic hedgehog protein is determined by size-exclusion chromatography.

8. A method for identifying compounds which increase or inhibit the production of oligomeric Sonic hedgehog protein (Shh) comprising expressing oligomeric Shh in the presence of a test compound and comparing an amount of oligomeric Shh expressed in the presence of the compound with an amount oligomeric Shh expressed in the absence of the compound.
